(19) United States
(12) Patent Application Publication
(10) Pub. No.: US 2024/0100268 A1
(43) Pub. Date: Mar. 28, 2024

(54) METHOD AND SYSTEM FOR EVALUATING INHALATION ADMINISTRATION

(71) Applicant: E-LINKCARE MEDITECH CO., LTD., Taizhou, Zhejiang (CN)

(72) Inventors: Tianxing WANG, Hangzhou (CN); Xijiang HU, Hangzhou (CN); Ziyi ZHANG, Hangzhou (CN); Liang JIANG, Hangzhou (CN); Qujiang LUO, Taizhou (CN)

(73) Assignee: E-LINKCARE MEDITECH CO., LTD., Taizhou, Zhejiang (CN)

(21) Appl. No.: 18/275,093

(22) PCT Filed: Jan. 27, 2022
(86) PCT No.: PCT/CN2022/074410

§ 371 Date: Jul. 31, 2023

(30) Foreign Application Priority Data
Jan. 29, 2021 (CN) 202110133307.9
Jan. 29, 2021 (CN) 202120269951.4

(51) Int. Cl. A61M 15/00

(52) U.S. Cl. CPC A61M 15/0065; A61M 2205/3334; A61M 2205/70; A61M 2230/46

(57) ABSTRACT

A method for evaluating inhalation administration and a system for evaluating inhalation administration, the system including a flow detector and an intelligent terminal. The method includes the following steps: 1) obtaining patient-related data of an inspiration flow-time curve; 2) according to an inspiration flow parameter of inhalation administration, obtaining a start time and an end time of each effective inspiration flow section on the inspiration flow-time curve; and 3) calculating an effective inspiration volume V_{effective} based on the inspiration flow-time curve and a series of time parameters tᵢ₁ and tᵢ₂ obtained. The method for evaluating inhalation administration and the system for evaluating inhalation administration provide a basis for medical workers to select an appropriate inhalation administration device for patients.

### FIELD OF TECHNOLOGY

The present invention relates to the field of inspiration evaluation, and more particularly to a system for evaluating inhalation administration and a method for evaluating inhalation administration.

### BACKGROUND

Inhalation therapy is a direct administration method through mouth and nose, taking respiratory tract and lungs as target organs. In the method, drug solution or powder is dispersed into droplets or particles by an inhalation administration device to be suspended in gas and inhaled into the respiratory tract and the lungs by human body to achieve therapeutic purposes. The inhalation therapy enables drugs to directly act on receptors on the surface of the respiratory tract, avoiding the obstruction of the first-pass effect of liver and the nervous system barrier, and directly reaching the disease site to play a therapeutic role. The inhalation therapy has the advantages of high local drug concentration, good bioavailability, rapid drug response, low systemic drug exposure, and mild adverse reaction. In recent years, with the continuous improvement of the development of inhalation administration device, the introduction of various new drugs and the improvement of preparation process, inhalation therapy has become more and more common in clinical application of respiratory diseases, and has become the preferred mode of drug administration for the prevention and treatment of respiratory diseases, such as asthma, chronic obstructive pulmonary disease (COPD) and other respiratory diseases.

Inspiration capacity is one of key factors of the inhalation therapy. The diameter and inspiration flow of drug particles are key factors of whether drugs can be inhaled and deposited effectively, so different drug preparations and different inhalation administration devices are required to have a different inspiration flow. Moreover, there is still a specified resistance in some inhalation administration devices, and patients must reach effective inspiration flow to overcome the internal resistance of the inhalation administration device, so that drug particles can be released therefrom, produce appropriate particles, and be effectively inhaled and deposited on a target site to exert their efficacy; otherwise, most of drug particles are only deposited in the mouth and throat, which seriously affects treatment and causes great waste. Different inhalation administration devices have different requirements and effects on inhalation flow due to different internal resistance; and the difference of internal resistance will inevitably affect the inspiration flow in different degrees. Movement modes of inhaled drugs in an airway include turbulence, laminar flow and Brownian motion. Turbulence is gas flow with many small swirls. An upper airway gas has a fast flow speed, which is easy to generate turbulence at the corner of the airway such as nose and throat. In a turbulent gas, drug particles with a particle size of no less than 5 μm and/or a flow rate of more than 60 L/min have high centrifugal force and are easy to hit and stay in the upper airway such as throat. Therefore, the inspiration flow is not as high as possible, but it should be determined according to the dosage form of the inhaled drug and the actual requirements of the inhalation administration device.

The principle of the inhalation administration device is to inhale drugs in the inhalation administration device into the respiratory system with the inspiration flow of the patients, thereby achieving the therapeutic effect through inhalation administration.

At present, inhaled drugs used clinically can be mainly divided into metered dose inhalation aerosol, dry powder inhalant (DPI), atomized liquid, soft mist inhalant, and pump atomization device mainly used for nasal inhalation. Table 1 shows some common inhalation administration device and effective and/or ideal inspiration flow parameters thereof.

TABLE 1
Common inhalation administration device and inspiration parameters thereof

| | | | | Effective | | Effective |
|---|---|---|---|---|---|---|
| | | | Inspiration | inspiration flow | Ideal | inspiration flow |
| Chinese | English | | resistance | Lower limit | inspiration flow | Upper limit |
| name | name | Type | Class | (L/min) | (L/min) | (L/min) |
| Aerosol | | pMDI | 0 | 10 | 30 | 60 |
| Respimat ® | Respimat ® | SMI | 0 | 10 | 30 | 60 |
| Breezhaler ® | Breezhaler ® | DPI | 1 | 50 | 50 | 90 |
| Turbohaler ® | Turbohaler ® | DPI | 3 | 30 | 60 | 90 |
| (Symicort) | (Symicort) | | | | | |
| Turbohaler ® | Turbohaler ® | DPI | 4 | 30 | 60 | 90 |
| (Pulmicort) | (Pulmicort) | | | | | |
| Handilaler ® | Handilaler ® | DPI | 5 | 20 | 30 | 90 |
| Accuhaler ® | Accuhaler ® | DPI | 2 | 30 | >60 | 90 |
| Diskus ® | Diskus ® | | | | | |
| Ellipta ® | Ellipta ® | DPI | 2 | 30 | 60 | 90 |

pMDI: pressurized metered-dose aerosol; SMI: soft mist inhalant; DPI: dry powder inhalant.

Studies have shown that with the increase of the severity of airway obstruction in COPD patients and children with asthma, the inspiration flow in each resistance state decreases. Therefore, although some inhalation administration devices can better produce uniform and fine drug particles, they are not suitable for patients who are frail or elderly, or suffer from severe airway obstruction because of their high internal resistance and high inspiration flow.

At present, a peak inspiration flow meter is commercially available and mechanically adjusts an aperture of a flow-limiting hole therein, which can simulate resistance states of different inhalation administration devices to evaluate the peak inspiration flow (PIF) of the patients and evaluate whether the inspiration flow of the patients matches with the inspiration flow of the inhalation administration device. However, such meter can only measure the peak inspiration flow (PIF) of the patients, but cannot evaluate the duration of effective inspiration flow, an effective inspiration volume, and other parameters, so the actual inhalation of effective drugs through the inhalation administration device cannot be accurately evaluated.

Therefore, it is of great clinical significance to quantitatively evaluate the inhalation administration for different patients and evaluate quantitative indicators of the inspiration flow of the patients under different resistance states of the inhalation administration device, which provides accurate quantitative reference for the selection of the appropriate inhalation administration device, improving the effectiveness of treatment and avoiding the waste of drugs.

### SUMMARY

In order to overcome the above problems and defects in the prior art, the present invention provides a method for evaluating inhalation administration and a system for evaluating inhalation administration.

An objective of that present invention is to provide a method for evaluating inhalation administration, and the method includes the following step: 1) using a flow detector and an inhalation administration device to be evaluated or a corresponding simulator to perform measurement according to inspiration requirements for the inhalation administration device, to obtain data of an inspiration flow-time curve (F-T); 2) according to an inspiration flow parameter of the inhalation administration device, obtaining a start time tᵢ₁ and an end time tᵢ₂ of each effective inspiration flow section on the inspiration flow-time curve; 3) calculating an effective inspiration volume V_{effective} based on the inspiration flow-time curve and a series of time parameters tᵢ₁ and tᵢ₂ obtained; and 4) evaluating an effective inhalation state of a patient using the inhalation administration device with the effective inspiration volume V_{effective} or indicators associated with the effective inspiration volume V_{effective}, to determine whether the inhalation administration device is applicable to the patient.

Further, the inspiration flow parameter includes a lower limit of effective inspiration flow.

Further, based on the lower limit of effective inspiration flow, a method for determining the effective inspiration flow section includes: taking a time point t₁₁ when inspiration flow reaches a preset lower limit of the effective inspiration flow for the first time as a start time of a first effective inspiration flow section; taking a time point t₁₂ when the inspiration flow drops to the preset lower limit of the effective inspiration flow for the first time as an end time of the first effective inspiration flow section; taking t₁₁ and t₁₂ sections in the inspiration flow-time curve as the effective inspiration flow section; and obtaining all effective inspiration flow sections by analogy.

Further, the inspiration flow parameter includes a lower limit of effective inspiration flow and an upper limit thereof.

Further, based on the lower limit of effective inspiration flow and the upper limit thereof, a method for determining the effective inspiration flow section includes: taking a time point t₁₁ when inspiration flow reaches a preset lower limit of the effective inspiration flow for the first time as a start time of a first effective inspiration flow section; taking a time point t₁₂ when the inspiration flow is beyond a range from the preset lower limit of the effective inspiration flow to a preset upper limit thereof for the first time as an end time of the first effective inspiration flow section; taking a time point t₂₁ when the inspiration flow starts to come into the range from the preset lower limit of the effective inspiration flow to the preset upper limit thereof for the second time as a start time of a second effective inspiration flow section; taking a time point t₂₂ when the inspiration flow is beyond the range from the preset lower limit of the effective inspiration flow to the preset upper limit thereof for the second time as an end time of the second effective inspiration flow section; and obtaining all effective inspiration flow sections by analogy.

Further, the indicator associated with the effective inspiration volume_{effective} is an effective inspiration volume ratio E_{effective}. The effective inspiration volume ratio E_{effective} is calculated by the following method: obtaining an inspiration start time t₀ and an inspiration end time tₓ on the inspiration flow-time curve; calculating a total inspiration volume Vₜₒₜₐₗ based on the inspiration flow-time curve and the obtained time parameters t₀ and tₓ; and then calculating the effective inspiration volume ratio E_{effective}. The total inspiration volume Vₜₒₜₐₗ and the effective inspiration volume ratio E_{effective} are calculated according to the following formula:

Vₜₒₜₐₗ=∫ₜ₀^{tx}f(t)dt

E_{effective}=(V_{effective}/Vₜₒₜₐₗ)*100%

where t denotes time, F(t) denotes a time function of inspiration flow, t₀ denotes an inspiration start time, and tₓ denotes an inspiration end time.

Further, the method further includes: using an effective inspiration duration T_{effective} and/or an average effective inspiration flow F_{effective}, where the effective inspiration duration T_{effective} and the average effective inspiration flow F_{effective} are calculated according to the following formula:

T_{effective}=Σᵢ₌₁ⁿ(ti2−ti1)

F_{effective}=V_{effective}/T_{effective}

i denotes the serial number of the effective inspiration flow section, and i=1 . . . n, where 1 denotes a first effective inspiration flow section and n denotes a last effective inspiration flow section; tᵢ₁ denotes a start time of the i(th) effective inspiration flow section; and tᵢ₂ denotes an end time of the i(th) effective inspiration flow section.

Further, the method further includes: determining a breath-holding duration, where a method for determining the breath-holding duration includes: obtaining a breath-holding end time tₕ on the inspiration flow-time curve and calculating the breath-holding duration (T_{breath-holding}) according to the following formula:

T_{breath-holding}=tₕ−tₓ

where tₕ denotes the breath-holding end time, and tₓ denotes an inspiration end time and a breath-holding start time.

Further, the method further includes: determining a peak inspiration flow (PIF), where a method for determining the peak inspiration flow includes: based on inspiration flow time data of the patient, obtaining a time tₚₑₐₖ when the inspiration flow reaches the peak inspiration flow; and obtaining a corresponding peak inspiration flow PIF.

Further, the effective inspiration volume V_{effective} is calculated according to the following formula:

V_{effective}=Σᵢ₌₁ⁿ(∫ₜᵢ₁^{ti2}F(t)dt)

t denotes time;

F(t) denotes a time function of inspiration flow;

i denotes the serial number of the effective inspiration flow section, and i=1 . . . n, where 1 denotes a first effective inspiration flow section and n denotes a last effective inspiration flow section;

tᵢ₁ denotes a start time of the i(th) effective inspiration flow section; and

tᵢ₂ denotes an end time of the i(th) effective inspiration flow section.

Further, the preset inspiration flow parameter of inhalation administration is a parameter provided by a commercial inhalation administration device or a parameter set by medical workers themselves, and the inspiration flow parameter is one or a combination of the lower limit of inspiration flow and the upper limit thereof.

The present invention provides a system for evaluating inhalation administration, and the system includes a flow detector 100 and an intelligent terminal 200, where the flow detector 100 detects patient-related data of an inspiration flow-time curve F-T with a time change, and the intelligent terminal 200 includes a data processing system and a method for evaluating inhalation administration; a wire or wireless connection is adopted between the flow detector and the intelligent terminal; and inspiration flow-time data detected by the flow detector 100 is uploaded to the intelligent terminal 200.

Further, the method for evaluating inhalation administration is adopted in the system for evaluating inhalation administration.

Further, the flow detector 100 is equipped with an intake resistance unit and includes different resistance gears to simulate the inhalation resistance of an existing commercial inhalation administration device.

Further, the intake resistance unit is equipped with a motor drive system, the parameter of the inhalation administration device to be evaluated and simulated is selected through the intelligent terminal 200, and a motor is automatically driven to set the intake resistance unit to a corresponding resistance gear.

Further, the flow detector 100 includes a flow detection unit, and can quantitatively measure flow data changed with time, and the principle type of flow detection is selected from one of a differential pressure flow meter, a rotor flow meter, a throttle flow meter, a turbine flow meter, a volumetric flow meter, a mass flow meter, an electromagnetic flow meter, and an ultrasonic flow meter.

Further, the intelligent terminal 200 is selected from smart phones, tablet computers, portable computers, desktop computers, hand-held computers, self-developed microcomputer terminals, or microcomputer terminals integrated with the flow detector 100.

Further, the intelligent terminal 200 further includes an evaluation guiding module for guiding an evaluation test of a patient, and the evaluation guiding module includes one or more guidance forms of sounds, characters, graphics and animations.

Further, the intelligent terminal 200 further includes a method for guiding the patient to perform inhalation evaluation in a corresponding inspiration way according to the requirements of the inhalation administration device selected for evaluation, and the characteristics of the inspiration way are selected from one or a combination not limited to an inspiration speed, an inspiration time, and a breath-holding duration.

More specifically, the system for evaluating inhalation administration includes a flow detector 100 and an intelligent terminal 200, where the flow detector 100 detects flow data changed with time, and the intelligent terminal 200 includes a data processing system and a method for evaluating inhalation administration based on the analysis of the inspiration flow-time curve (F-T); a wire or wireless connection is adopted between the flow detector 100 and the intelligent terminal 200 to perform interaction. The method for evaluating inhalation administration includes the following step: 1) using a flow detector and an inhalation administration device to be evaluated or a corresponding simulator to perform measurement according to inspiration requirements for the inhalation administration device, to obtain data of an inspiration flow-time curve (F-T); 2) according to an inspiration flow parameter of the inhalation administration device, obtaining a start time tᵢ₁ and an end time tᵢ₂ of each effective inspiration flow section on the inspiration flow-time curve; 3) calculating an effective inspiration volume V_{effective} based on the inspiration flow-time curve and a series of time parameters tᵢ₁ and tᵢ₂ obtained; and 4) evaluating an effective inhalation state of a patient using the inhalation administration device with the effective inspiration volume V_{effective} or indicators associated with the effective inspiration volume V_{effective}, to determine whether the inhalation administration device is applicable to the patient. The method may further includes: obtaining other time parameters based on the inspiration flow-time curve: the inspiration start time t₀, the inspiration end time tₓ, the breath-holding end time tₕ, and the peak inspiration flow time tₚₑₐₖ; then calculating more evaluation indicators, including the effective inspiration duration (T_{effective}), the average effective inspiration flow (F_{effective}), the total inspiration volume (Vₜₒₜₐₗ), the effective inspiration volume ratio (E_{effective}), the breath-holding duration (T_{breath-holding}), the peak inspiration flow (PIF), and the like. Through one or more of these indicators, the medical workers can evaluate the effectiveness of inhalation administration for patients.

As shown in FIG. 1, the flow detector 100 includes a ventilation inlet 110, a flow detection unit 120, and a human body inspiration inlet 130. Airflow enters from the ventilation inlet 110, passes through the flow detection unit 120, and then is inhaled into a human body through the human body inspiration inlet 130. In FIG. 1, there is an interactive arrow between the flow detector 100 and the intelligent terminal 200, indicating that data detected by the flow detector 100 can be uploaded to the intelligent terminal 200. In addition, in some embodiments, the intelligent terminal 200 is combined with a detection control module to send an operating instruction to the flow detector 100.

In some embodiments, the ventilation inlet 110 is equipped with a resistance adjusting device to simulate the internal resistance of different inhalation administration devices. During the evaluation, the resistance gear can be set according to public information of the inhalation administration devices currently on the market, or can be also determined through the measurement of the internal resistance data of these devices. For the inhalation administration device with substantively no resistance, such as aerosol, the resistance gear can be set to 0 during the evaluation. In another embodiment, the ventilation inlet 110 is equipped with different adapters and connected with different inhalation administration devices to evaluate the actual use of the inhalation administration device by a patient.

The human body inhalation inlet 130 can be designed as a human body inhalation adapter in the form of a mouthpiece, a mask and others for the adaptive selection of oral inhalation or nasal inhalation to the human body. More preferably, in some embodiments, the human body inhalation adapter may be designed as a replaceable single-use device provided with a filtration component to avoid the risk of cross contamination when the inhalation administration device is used by a plurality of persons.

The flow detection unit 120 can quantitatively measure flow data changed with time. The principle type of flow detection includes, but is not limited to, a differential pressure flow meter, a rotor flow meter, a throttle flow meter, a turbine flow meter, a volumetric flow meter, a mass flow meter, an electromagnetic flow meter, and an ultrasonic flow meter. The flow detection unit may be a commercially available digital flow meter or may be designed according to the detection principle. The flow-time (F-T) data detected by the flow detection unit should be uploaded to the intelligent terminal 200 in a wired or wireless transmission way.

In some embodiments, the flow detector 100 can be equipped with a detection control module, and the detection control module includes a software part and a hardware part. The detection control module automatically controls the flow detector 100 to start detecting and identifying the signal of inspiration start after receiving a detection start instruction, collects data of inspiration flow and time in real time, and automatically identifies information of inspiration end, breath holding, and breath-holding end. After the detection, the detected flow-time data can be stored or/and transmitted to an evaluation software. The detection control module of the flow detector 100 can be designed integrally with the flow detector 100, or may be also set separately and connected to the intelligent terminal 200 and the flow detector 100. The detection control module can also be partially included in the intelligent terminal 200, that is, the software part of the detection control module is included in the intelligent terminal 200, and the hardware part thereof is provided on the flow detector 100 or provided separately.

In other embodiments, the detection of the flow detector 100 can also be manually started and ended, and then the intelligent terminal 200 can analyze the flow-time curve (F-T) to determine inspiration start time, inspiration end time, and breath-holding end time. Similar to the automatic control of the detection control module, a method for identifying a time point by the intelligent terminal 200 includes: taking a time when the inspiration flow starts to rise from 0 as an inspiration start time point t₀; and taking a time when the inspiration flow drops to 0 as an inspiration end time point tₓ. The inspiration end also means the breath-holding start. During the breath holding, the flow is substantively 0, and a time point when the flow is from 0 to expiration flow is the time point tₕ of the breath holding end.

Before inspiration evaluation, a patient can select the appropriate resistance gear of the ventilation inlet 110 according to the resistance of the inhalation administration device to be evaluated. After preparation, the inspiration evaluation is made according to the requirements of the inhalation administration device to be evaluated. Breath holding is done after the inspiration end; and expiration is done after breath holding is done according to the requirements for the breath-holding time or cannot be done again. The flow detector 100 detects the flow-time data during the evaluation and uploads such data to the intelligent terminal 200.

The intelligent terminal 200 includes a data processing system and a method for evaluating inhalation administration based on the analysis of the inspiration flow-time curve (F-T). Users can select the flow parameter information of the inhalation administration device corresponding to the inhalation resistance through input or selection or automatically according to the gear of the inhalation administration device, such as one or a combination of a lower limit of effective inspiration flow and an upper limit thereof. Then, the flow-time curve is compared and analyzed to obtain the inspiration start time t₀, the start time tᵢ₁ and end time tᵢ₂ of each effective inhalation flow section, the time tₚₑₐₖ when the inspiration flow reaches the peak inspiration flow, the inspiration end time tₓ, and the breath-holding end time tₕ, and obtain the peak inspiration flow (PIF).

FIG. 2a-FIG. 2b are examples of analysis of the flow-time curve. In the flow-time curve in the figure, the inspiration start time point t₀ has been automatically set as a time axis zero point; the intelligent terminal 200 can detect decrease in the inspiration flow to detect a time point when the inspiration flow drops to 0, and the time point is taken as the inspiration end time point tₓ; the intelligent terminal 200 can detect the inspiration end to detect a time point when the inspiration flow is changed from 0 to the expiration flow, and the time point is the breath-holding end time tₕ; and the intelligent terminal 200 can further detect the inspiration flow to obtain the peak inspiration flow (PIF) and the corresponding peak inspiration flow inspiration time tₚₑₐₖ.

The inhalation flow parameter of the inhalation administration device corresponding to the example in FIG. 2a only includes the lower limit of effective inspiration flow and is 30 L/min (as shown by the horizontal dashed line in the figure). The intelligent terminal 200 can compare the inhalation flow with the lower limit of effective inspiration flow from a zero time t₀ to detect the time point t₁₁ when the inspiration flow reaches the lower limit of effective inspiration flow for the first time, and the time point is the start time of the first effective inspiration flow section; and the intelligent terminal 200 continues to perform such comparison along a time axis to detect the time point t₁₂ when the inspiration flow drops to the lower limit of effective inspiration flow for the first time, and the time point is the end time of the first effective inspiration flow section. Then, the intelligent terminal 200 continues to perform such comparison along the time axis to detect the start time tᵢ₁ and end time tᵢ₂ of all effective inhalation flow sections until the inspiration end time tₓ. As shown in FIG. 2a, a shaded area is the effective inspiration flow section. The inhalation flow parameter of the inhalation administration device corresponding to the example in FIG. 2b includes the lower limit of effective inspiration flow of 30 L/min (as shown by the upper horizontal dashed line in the figure), the ideal inspiration flow of 60 L/min (shown by the middle horizontal dashed line in the figure), and the upper limit of effective inspiration flow of 90 L/min (shown by the lower horizontal dashed line in the figure). Similar to the example in FIG. 2a, the intelligent terminal 200 can compare the inhalation flow with the lower limit of effective inspiration flow and the upper limit of effective inspiration flow from the zero time t₀ to detect the time point t₁₁ when the inspiration flow reaches the lower limit of effective inspiration flow, and the time point is the start time of the first effective inspiration flow section; and the intelligent terminal 200 continues to perform such comparison along the time axis to detect the time point t₁₂ when the inspiration flow exceeds the interval from the lower limit of effective inspiration flow to the upper limit of effective inspiration flow, and the time point is the end time of the first effective inspiration flow section. Then, the intelligent terminal 200 continues to perform such comparison along the time axis to detect the time point t₂₁ when the inspiration flow starts to enter the interval from the lower limit of effective inspiration flow to the upper limit of effective inspiration flow for the second time, and the time point is the start time of the second effective inspiration flow section; and the intelligent terminal 200 continues to perform such comparison along the time axis to detect the time point t₂₂ when the inspiration flow exceeds the interval from the lower limit of effective inspiration flow to the upper limit of effective inspiration flow for the second time, and the time point is the end time of the second effective inspiration flow section. Then, the intelligent terminal 200 continues to perform such comparison along the time axis to detect the start time tᵢ₁ and end time tᵢ₂ of all effective inhalation flow sections until the inspiration end time tₓ. As shown in FIG. 2b, a shaded area is the effective inspiration flow section.

Substantially, the examples in FIG. 2a and FIG. 2b share a way to detect an effective inhalation flow section, but are different in terms of determining parameters of the effective inhalation flow section: the example in FIG. 2a denotes the effective inspiration flow section when the inspiration flow is greater than the lower limit of effective inspiration flow, and the example in FIG. 2b denotes the effective inspiration flow section when the inspiration flow is within the interval from the lower limit of effective inspiration flow to the upper limit of effective inspiration flow.

The flow-time curve (F-T) of human inspiration is normal, that is, the inspiration flow is low at the beginning of inspiration, rises rapidly and reaches the peak inspiration flow, and then begins to decline until it drops to zero, and the inspiration is ended. After the inspiration, the human body needs to perform breath holding according to the requirements of some inhalation administration devices to increase the deposition rate of drugs; and when the human body can no longer perform breath holding, it will exhale breath and end up breath holding. Based on the ideal inhalation flow-time curve, the effective inhalation flow section is usually not too complicated. There are 1-2 effective inhalation flow sections according to different flow parameters of the inhalation administration device. However, different subjects are different in physical conditions and familiarity with skills of detection coordination. In fact, during the inspiration evaluation, it is possible to encounter an example where the inspiration flow-time curve fluctuates. For example, the inspiration flow is likely to repeatedly rise and fall in a rising process before reaching the peak inspiration flow; in a falling process after the inspiration flow reaches the peak inspiration flow, the inspiration flow repeatedly rises and falls before falling to zero. Therefore, in this case, the effective inhalation flow section will be relatively complicated and may be divided into a plurality of sections.

In addition, in some embodiments, in order to allow the medical workers to evaluate the inhalation administration of patients more carefully or flexibly, the intelligent terminal 200 can set the upper limit of the ideal inspiration flow and the lower limit thereof, for example, when the ideal inspiration flow is 60 L/min, the upper limit and the lower limit are set to 10% and −10% respectively, so the lower limit of the ideal inspiration flow is 54 L/min and the upper limit of the ideal inspiration flow is 66 L/min. Accordingly, the intelligent terminal 200 can obtain the start time tⱼ₁ and end time tⱼ₂ of each ideal inhalation flow section using an acquisition method similar to that of the effective inhalation flow section.

Then, the intelligent terminal 200 can calculate the effective inspiration duration (T_{effective}), the effective inspiration volume (V_{effective}), the total inspiration volume (Vₜₒₜₐₗ), the effective inspiration volume ratio (E_{effective}), the breath-holding duration (T_{breath-holding}) and other indicator parameters based on the obtained time parameters and the flow-time curve (F-T). The calculation formulas are as follows.

T_{effective}=Σᵢ₌₁ⁿ(ti2−ti1) Formula 1:

V_{effective}=Σᵢ₌₁ⁿ(∫ₜᵢ₁^{ti2}F(t)dt) Formula 2:

Vₜₒₜₐₗ=∫ₜ₀^{tx}F(t)dt Formula 3:

E_{effective}=(V_{effective}/Vₜₒₜₐₗ)*100% Formula 4:

T_{breath-holding}=tₕ−tₓ Formula 5:

t denotes time;

F(t) denotes a time function of inspiration flow;

i denotes the serial number of the effective inspiration flow section, and i=1 . . . n, where 1 denotes a first effective inspiration flow section and n denotes a last effective inspiration flow section;

tᵢ₁ denotes a start time of the i(th) effective inspiration flow section; and

tᵢ₂ denotes an end time of the i(th) effective inspiration flow section;

t₀ denotes the inspiration start time;

tₓ denotes an inspiration end time and a breath-holding start time; and

tₕ denotes the breath-holding end time.

The above calculation methods are only examples in some embodiments of the present invention, and some well-known calculation methods within the spirit of the present invention are all protected by the present invention, for example, calculating and accumulating the areas of approximate trapezoids by subdividing time intervals for integral substitution.

The indicator parameters and the effective inspiration duration (T_{effective}) can be used to evaluate the effective inhalation time when patients use the inhalation administration device. The effective inspiration volume (V_{effective}) can be used to evaluate the effective inhalation flow volume when the patients use the inhalation administration device. The effective inhalation volume ratio (E_{effective}) can be used to evaluate the ratio of the effective inhalation flow volume to the total inhalation flow volume when the patients use the inhalation administration device, which can evaluate the effective ratio and waste ratio of drug inhalation. The breath-holding duration (T_{breath-holding}) can be used to evaluate the time allowed for drug deposition after inhalation.

Based on the indicator parameters, the average effective inspiration flow (F_{effective}) can also be calculated and can be compared with the ideal inspiration flow of the inhalation administration device. The calculation formula is as follows.

F_{effective}=V_{effective}/T_{effective} Formula 6:

Similarly, in an embodiment where the upper limit of the ideal inhalation flow and the lower limit thereof can be set, the ideal inspiration volume (V_{ideal}) and the ideal inspiration volume ratio (E_{ideal}) can be calculated according to the following formula.

V_{ideal}=Σⱼ₌₁ⁿ(∫ₜⱼ₁^{tj2}F(t)dt) Formula 7:

E_{effective}=(V_{effective}/Vₜₒₜₐₗ)*100% Formula 8:

j denotes the serial number of the ideal inspiration flow section, and j=1 . . . n, where 1 denotes a first ideal inspiration flow section and n denotes a last ideal inspiration flow section;

tⱼ₁ denotes a start time of the j(th) ideal inspiration flow section; and

tⱼ₂ denotes an end time of the j(th) ideal inspiration flow section.

In some embodiments, the intelligent terminal 200 can be designed integrally with the flow detector 100, and the intelligent terminal 200 can further include a display module, an input module, a data processing system, and a method for evaluating inhalation administration based on the analysis of the inspiration flow-time curve (F-T), can display an interactive interface thereof in the display module, and can perform human-computer interaction through a touch screen or an input module/button.

In other embodiments, the intelligent terminal 200 may be another computer terminal. The computer terminal includes, but is not limited to, smart phones, tablet computers, portable computers, desktop computers, hand-held computers, self-developed microcomputer terminals, and the like. The flow detector 100 can interact with the intelligent terminal 200 in a wired or wireless way. In some application scenarios, data of the system for evaluating inhalation administration can also be uploaded to a server of a cloud platform in a wired or wireless way, facilitating home self-evaluation by patients and giving remote guidance by doctors or immediate superior medical institutions to subordinate medical institutions thereof.

In some embodiments, the intelligent terminal 200 may include the software part of the detection control module of the flow detector 100. Through the software part of the detection control module, the intelligent terminal 200 can set the parameters for evaluation test, and control the flow detector 100 to operate in a set way: for example, the corresponding inhalation resistance gear of the flow detector 100 is automatically set according to the parameters of the inhalation administration device selected for evaluation. During evaluation, the flow detector 100 is driven to a state to be evaluated, and automatically identifies the inspiration start time, the inspiration end time, and the breath-holding end time and automatically ends up the evaluation. In addition, during the evaluation, evaluation status information fed back by the flow detector 100 is obtained in real time.

In some embodiments, the intelligent terminal 200 is provided with an evaluation guiding module that guides a user to perform detection, and the guiding module can be one or more combinations of characters, animations, pictures, and sounds. For different inhalation drug dosage forms and inhalation administration devices, requirements for inhalation operation of patients are also different, including inspiration speed, time, breath-holding duration, and the like. Necessary guidance can improve the effectiveness of detection.

For example, for two types of active spraying devices pMDI and SMI, inspiration flow of the patients does not affect aerosol properties, and slow and deep inspiration helps to inhale more drugs, improve the pulmonary deposition rate, and reduce the oropharyngeal deposition. The specific requirements are that: the slow and deep inspiration is performed after deep expiration, and the inspiration speed is generally about 30 L/min and the ideal flow of the use of the active spraying devices.

For DPI inhalation administration devices and drugs, drug powder needs to be depolymerized into fine drug particles depending on the internal resistance of the device and the turbulence generated through the active inhalation of patients, so the inspiration volume and inspiration flow of the patients affect the delivery rate of DPI and the size and movement speed of delivered drug particles thereof. The patients inhale with large volume and fast speed, which helps to improve the drug delivery rate of DPI and the proportion of small particles thereof and improve the curative effect. Therefore, the patients need to inhale quickly and forcefully when using DPI.

In addition, the breath-holding ability of the patients also has a significant impact on the effect of deposition of drugs in a small airway, so the patients usually need to hold their breath for a period of time (about 10 s) after drug inhalation to facilitate the deposition of the drugs in the small airway.

The evaluation guiding module can be combined with the detection control module of the flow detector 100 to give timely guidance to the users through real-time acquisition of evaluation status information fed back by the flow detector 100 in real time. For example, before the evaluation, the users are prompted on how to perform the inspiration; at the end of the inspiration, the breath holding and the breath holding duration are reminded to the users.

In some embodiments, the intelligent terminal 200 is further provided with a patient information management module, which can input or import information such as name, age, gender, and medical history of the patients. In some application scenarios, evaluation data of some embodiments may be accessed to an LIS or HIS system of a hospital.

In some embodiments, the intelligent terminal 200 may also be externally connected or externally connected to a printing device, so that an evaluation report can be printed out as needed.

In some embodiments, the intelligent terminal 200 may set a method for evaluation test as multiple modes, and some parameters between multiple evaluations and detections should be within a range, for example, the peak inspiration flow (PIF) should be within 10%. An optimal value, an average value, or a user-selected value may be taken for the flow-time curves for the multiple modes, and an evaluation software evaluates and analyzes the final flow-time curves; alternatively, the evaluation software can analyze the flow-time curves for a single mode separately, and/or give the optimal value or average value at the same time.

In some embodiments, the system for evaluating inhalation administration can be realized based on the existing flow detector, combined with some adaptive structural modifications, and appropriate software upgrade including a method for processing and analyzing the inhalation administration data. The flow detector is a pulmonary function analyzer.

In some embodiments, the system for evaluating inhalation administration can also be used as a tool for patients to perform training on inhalation administration. The system for evaluating inhalation administration can be further equipped with a training module to improve the efficiency of inhalation administration for the efficiency of inhalation administration for patients through the training.

The method and system for inhalation administration proposed by the present invention based on the effective inspiration volume V_{effective} provide a more accurate determining basis for medical staff to select a suitable inhalation administration device for patients. Meanwhile, the method can be further combined with more indicator parameters, including the effective inspiration duration (T_{effective}), the effective inspiration volume (V_{effective}), the average effective inspiration flow (F_{effective}), the total inspiration volume (Vₜₒₜₐₗ), the effective inspiration volume ratio (E_{effective}), the breath-holding duration (T_{breath-holding}), the peak inspiration flow (PIF), and the like. In addition, the method can provide a basis for medical workers to select an appropriate inhalation administration device for patients according to one or more of these indicator parameters. Therefore, the inhalation administration for different patients is quantitatively evaluated and quantitative indicators of the inspiration flow of the patients under different resistance states of the inhalation administration device are evaluated. This provides accurate quantitative reference for the selection of the appropriate inhalation administration device, avoiding the waste of drugs.

### BRIEF DESCRIPTION OF THE DRAWINGS

FIG. 1 shows a schematic block diagram of a system for evaluating inhalation administration according to the present invention.

FIG. 2a shows an example diagram 1 of analysis of a flow-time curve (F-T).

FIG. 2b shows an example diagram 2 of analysis of a flow-time curve (F-T).

FIG. 3 shows a schematic diagram of a system for evaluating inhalation administration according to an embodiment.

FIG. 4 shows a view of a ventilation inlet side of a flow detector according to an embodiment.

FIG. 5 shows an information table and a new example of an inhalation administration device of a display interface of an intelligent terminal.

FIG. 6 shows a flow diagram of evaluation of inhalation administration according to an embodiment.

FIG. 7 shows a flow-time curve and an analysis diagram of a test example 1.

FIG. 8 shows a flow-time curve and an analysis diagram of a test example 2.

FIG. 9 shows a flow-time curve and an analysis diagram of a test example 3.

FIG. 10 shows a flow-time curve and an analysis diagram of a test example 4.

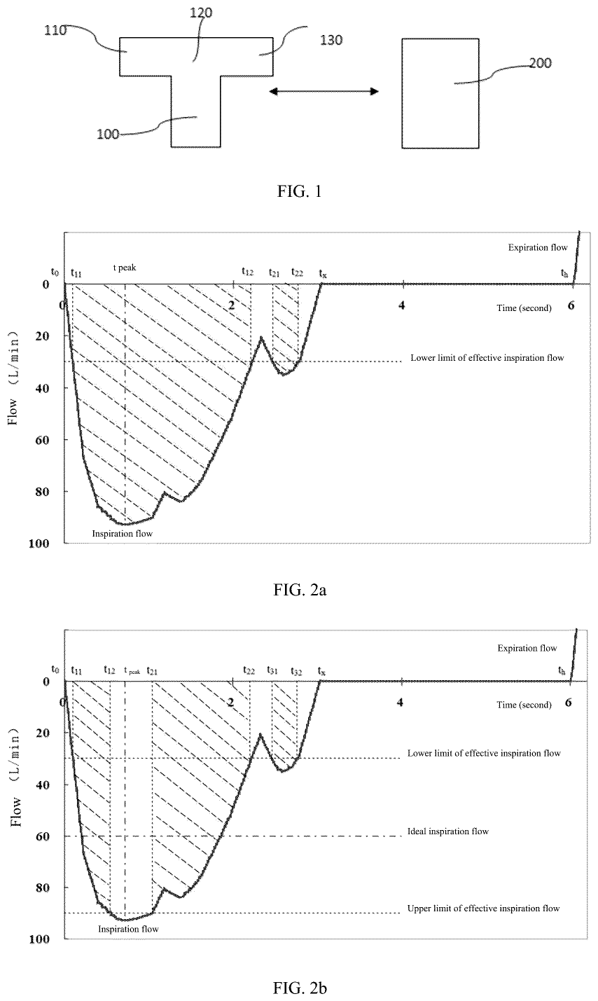

### DESCRIPTION OF THE EMBODIMENTS

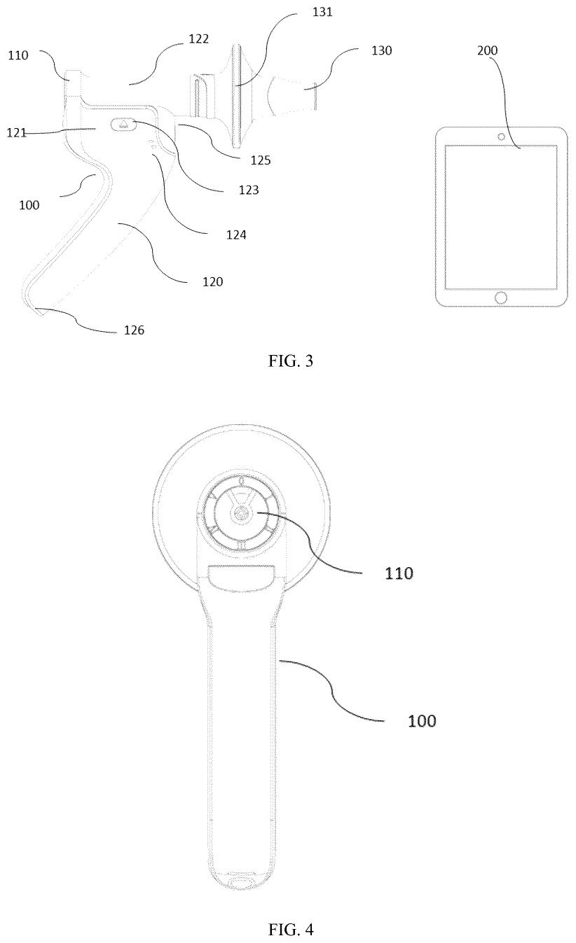

FIG. 3 shows a schematic diagram of a system for evaluating inhalation administration according to the present invention. A flow detector 100 is based on the detection principle of a differential pressure flow meter; an intelligent terminal 200 is a terminal such as a computer, a tablet computer or a smart phone; and the flow detector and the intelligent terminal 200 can be connected in a wired or wireless way (Bluetooth).

A ventilation inlet 110 of the flow detector 100 is provided with an intake resistance device (as shown in FIG. 4) having different resistance gears (there are six gears in the embodiment, as shown in Table 2); and the intake resistance device is equipped with a motor drive module to simulate the internal resistance of different inhalation administration devices. The intelligent terminal 200 can select the inhalation administration device for simulated evaluation to automatically adjust a motor instruction to a corresponding intake resistance gear. The design of automatic adjustment of the resistance gears can avoid the risk of forgetting or wrong adjustment due to manual adjustment.

TABLE 2
Description of intake resistance gear for embodiments

| Inspiration | Display of inspiration | Class |
|---|---|---|
| impedance class | impedance class | description |
| 0 | 0 | Very low or none |
| 1 | I | Low |
| 2 | II | Medium-low |
| 3 | III | Medium |
| 4 | IV | Medium-high |
| 5 | V | High |

A flow detection unit 120 includes a differential pressure flow detection tube 122 and a main body 121 of the flow detection unit. The main body 121 of the flow detection unit is equipped with a flow detection sensor, a hardware part of a detection control module of the flow detector 100, a wireless transmission module (Bluetooth and/or 2G/4G/5G module), a battery, and the like. A housing of the main body 121 of the flow detection unit is equipped with a wired connection port 126 (such as USB), an on-off button 125, an operating status indicator 124, and a fastener removal button 123 of a flow detection tube. The flow detection tube 122 of the embodiment is designed to be detachable and replaceable, and the flow detection tube 122 can be easily removed through the fastener removal button 123. The differential pressure flow detection tube 122 includes a high pressure tapping hole and a low pressure tapping hole, and the high pressure tapping hole and the low pressure tapping hole are respectively connected with a flow monitoring sensor through an airway.

A mouthpiece 130 is adopted at a human body inspiration inlet, and the mouthpiece 130 and the flow detection tube 122 are closely connected through a bayonet and interference fit, and are designed to be detachable. A filtration layer 131 is arranged in the mouthpiece to prevent bacteria and dust from being inhaled into the human body, and prevent exhaled gas from polluting the flow detection tube 122, thus eliminating the risk of cross contamination.

A software part of the detection control module of the flow detector 100 is integrated into the intelligent terminal 200; and the intelligent terminal 200 includes, but is not limited to, smart phones, tablet computers, portable computers, desktop computers, hand-held computers, self-developed microcomputer terminals, and the like.

The intelligent terminal 200 includes a patient information management module. Management information includes name, medical record number, gender, age, height, weight, medical history, and the like of patients. Some predicted values need be calculated according to the specific information of the patients. Patient information can be entered manually or imported through a system, such as an LIS or HIS system of a hospital. Before evaluation, a patient is selected or new patient information is entered, and then a next step is proceeded.

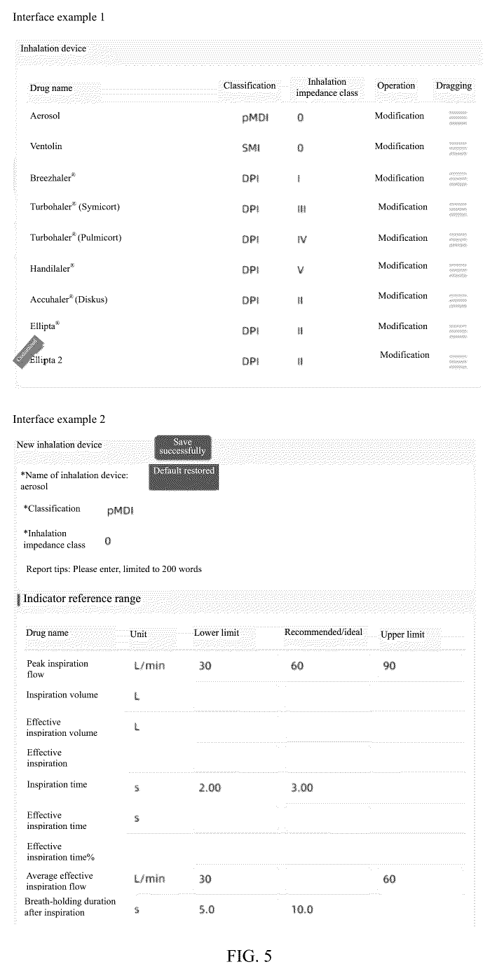

The intelligent terminal 200 includes an information table of the inhalation administration device. Information of some inhalation administration devices commonly used in the market is in advance stored, and can be added or modified, as shown in FIG. 5. The information table includes indicators such as name, brand model, internal resistance gear information, lower limit of effective inspiration flow, ideal inspiration flow, upper limit of effective inspiration flow, or some of indicators, and displays information for some or all of the indicators according to the actual situation of the inhalation administration device. A user can also add other types of inhalation administration devices as needed and enter information corresponding to the indicator items; and medical workers can also customize indicator parameters based on their insights and experience in use of the inhalation administration devices.

Before the evaluation, the flow detector 100 is connected to the intelligent terminal 200 and validated for powering on; the user can select an inhalation administration device to be evaluated through display and interaction interfaces of the intelligent terminal 200; the intelligent terminal 200 gives an instruction to the flow detector 100; and the flow detector 100 automatically sets the resistance gear to the resistance gear corresponding to the inhalation administration device to be evaluated through a drive motor. The inhalation administration device to be evaluated is selected or information thereof is entered, and a next step is proceeded. The intelligent terminal 200 gives an option of an evaluation frequency mode, such as 1 times, 2 times, and 3 times, with 1 times as a default. If a multi-evaluation mode is selected, an option for an acceptance standard of a deviation between multiple evaluations, such as a deviation of peak inspiration flow and a deviation of effective inspiration volume, will be given and can be selected and entered as needed.

In addition, the intelligent terminal 200 gives evaluation and analysis methods for the user to select: if the selected inhalation administration device to be evaluated only includes information of the lower limit of effective inspiration flow, only mode 1 can be selected by default for the analysis method, that is, the analysis method that the effective inspiration volume is based on the lower limit of effective inspiration flow; if the selected inhalation administration device to be evaluated includes information of the lower limit and upper limit of effective inspiration flow, only mode 2 can be selected by default for the analysis method, that is, the analysis method that the effective inspiration volume is based on the lower limit and upper limit of effective inspiration flow; and the user can also manually select the mode 1 to perform evaluation by ignoring the upper limit of effective inspiration flow. If the selected inhalation administration device to be evaluated includes information of the ideal inspiration flow, software will have an option to select whether the ideal inspiration volume is evaluated and give the range of the ideal inspiration flow; if the ideal inspiration volume is evaluated, the ideal inspiration volume V ideal and an ideal inspiration volume ratio E_{ideal} will be analyzed.

The intelligent terminal 200 also includes an evaluation guiding module, and when the user gives an instruction from the intelligent terminal 200 to start evaluation test, the software will give one or more prompts, such as pictures, animations, characters, and sounds, to guide the user or patient to perform the evaluation, and the software will give guidance for a next operation in a timely manner according to the evaluation status information in real time fed back by the flow detector 100.

The flow detector 100 detects the flow-time data during the evaluation and in real time uploads such data to the intelligent terminal 200, displaying the dynamic flow-time curve. If the user selects a multi-detection mode, the intelligent terminal 200 will give a prompt for next detection after one detection.

After the evaluation, the intelligent terminal 200 will analyze detection results according to the selected evaluation and analysis methods, and then display a result interface. The user can select and give a detection curve, a reporting indicator, and the like of a report according the result interface to give a final evaluation report, including:
Selecting an evaluation curve to analyze and give a report: if only one evaluation is conducted, the evaluation is defaulted; if multiple evaluations are conducted, the user can select an optimal evaluation curve, select the evaluation curve at will, take an average value between several evaluations, and make separate statistics for each evaluation and give the average value and or/and the optimal value, and other modes. If a deviation between results of the several evaluations exceeds a set standard, the intelligent terminal 200 gives a warning and a prompt as to whether the evaluation is supplemented.
Selecting indicators given by the report: according to actual information of the inhalation administration device to be evaluated, a selectable option for the evaluation report is given, including an effective inspiration duration (T_{effective}), the effective inspiration volume (V_{effective}), an average effective inspiration flow (F_{effective}), a total inspiration volume (Vₜₒₜₐₗ), an effective inspiration volume ratio ((E_{effective}), the ideal inspiration volume (V_{ideal}), the ideal inspiration volume ratio (V_{ideal}), a breath-holding duration (T_{breath-holding}), a peak inspiration flow (PIF), and the like.

After a report option is selected, the intelligent terminal 200 will generate an electronic evaluation report, and the report can be printed out as needed.

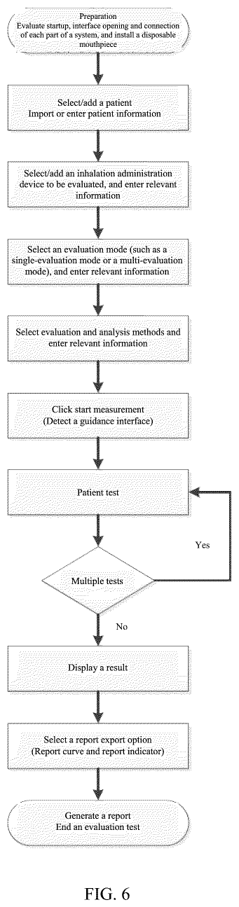

Based on the system for evaluating inhalation administration in the above embodiment, a method for evaluating inhalation administration is provided below, with an evaluation test process as shown in FIG. 6 and described below:
1) Preparation: opening an evaluation interface of the intelligent terminal 200 and turning on the flow detector 100, to ensure that the intelligent terminal 200 and the flow detector 100 are connected in a wired (USB cable) or wireless (Bluetooth) manner, and the intelligent terminal 200 can detect the flow detector 100. A disposable mouthpiece 130 is installed on the flow detector 100.
2) Selecting or adding patients, and importing or entering patient information, including name, medical record number, gender, age, height, weight, medical history information, and the like.
3) Selecting, modifying, adding, or entering the inhalation administration device to be evaluated and related information thereof for patients, such as: name, brand model, resistance gear, lower limit of effective inspiration flow, optimal inspiration flow, upper limit of effective inspiration flow, breath-holding time, and the like of the inhalation administration device.
4) Selecting an evaluation mode, such as a single-evaluation mode or a multi-evaluation mode. If the multi-evaluation mode is selected, the evaluation frequency and the acceptance standard of the deviation between multiple evaluations are entered, such as the deviation of peak inspiration flow and the deviation of effective inspiration volume, will be selected and entered as needed.
5) Selecting the evaluation and analysis methods. The system will match the evaluation and analysis methods by default according to the information of the selected inhalation inspiration device before detection, such as:
If the selected inhalation administration device to be evaluate only includes the information of the lower limit of effective inspiration flow, only mode 1 can be selected by default for the analysis method, that is, the analysis method that the effective inspiration volume is based on the lower limit of effective inspiration flow;
If the selected inhalation administration device to be evaluated includes information of the lower limit and upper limit of effective inspiration flow, only mode 2 can be selected by default for the analysis method, that is, the analysis method that the effective inspiration volume is based on the lower limit and upper limit of effective inspiration flow; and the user can also manually select the mode 1.
If the selected inhalation administration device to be evaluated includes information of the ideal inspiration flow, software will provide the user with an option to select whether the ideal inspiration volume is evaluated; if the ideal inspiration volume is evaluated, the user needs to input the ideal inspiration flow range.
6) Clicking start measurement: the intelligent terminal 200 will give an instruction to adjust the resistance gear of the flow detector 100 to the gear corresponding to the selected inhalation administration device, and then the intelligent terminal 200 will guide the patients to perform inspiration evaluation. According to the type of the selected inhalation administration device, the intelligent terminal 200 will guide the patients to adopt different inspiration modes as follows.
DPI: The patients try to take breaths as quickly as possible.
pMDI/SMI: The patients take slow and deep breaths.
7) Before the evaluation, the patients should exhale as much as possible, then clench their mouthpiece and perform inspiration evaluation under the guidance of the intelligent terminal 200. The intelligent terminal 200 can detect the change of inspiration flow, automatically identify an inspiration zero time, and transmit flow-time data to the intelligent terminal 200 in real time. After inspiration, the intelligent terminal 200 can detect that the inspiration flow drops to zero, thus identifying an inspiration end time point and a breath-holding start time point. The patients should hold their breath according to a prompt of the intelligent terminal 200 (the breath-holding time is set according to requirements of the inhalation administration device), and the intelligent terminal 200 can identify the end of breath holding by detecting expiration flow and stop the detection. After the patients hold their breath for more than the breath-holding time, the intelligent terminal 200 can also stop the detection.
8) If the multi-evaluation mode is selected, the intelligent terminal 200 will pop up a prompt for next detection; the user and the patient repeat tests in 6) to 7) until the test is completed.
9) After the evaluation test, the result interface will be given according to the test result and the selected analysis method.
10) The evaluation curve of the report can be selected.

If only one evaluation is conducted, the evaluation is defaulted; if multiple evaluations are conducted, the user can select an optimal evaluation curve, select the evaluation curve at will, take an average value between several evaluations, and make separate statistics for each evaluation and give the average value and or/and the optimal value, and other modes. If a deviation between results of the several evaluations exceeds a set standard, the software gives a warning and a prompt as to whether the evaluation is supplemented.
11) Indicators given in the report can be selected

According to actual information of the inhalation administration device to be evaluated, a selectable option for the evaluation report is given, including an effective inspiration duration (T_{effective}), the effective inspiration volume (V_{effective}), an average effective inspiration flow (F_{effective}), a total inspiration volume (Vₜₒₜₐₗ), an effective inspiration volume ratio ((E_{effective}), the ideal inspiration volume (V_{ideal}), the ideal inspiration volume ratio (V_{ideal}), a breath-holding duration (T_{breath-holding}), a peak inspiration flow (PIF), and the like.
12) The intelligent terminal 200 generates the report, and the evaluation test is ended.

The system for evaluating inhalation administration according to the present invention can also be applied to the training of inhalation administration of the patients to train them to achieve the most appropriate inspiration flow of medication, thereby improving the quality of medication.

The following describes the system for evaluating inhalation administration and the method for evaluating inhalation administration based on the above embodiments, and the user selects different resistance simulation gears and different analysis modes of the inhalation administration devices.

### TEST EXAMPLE 1

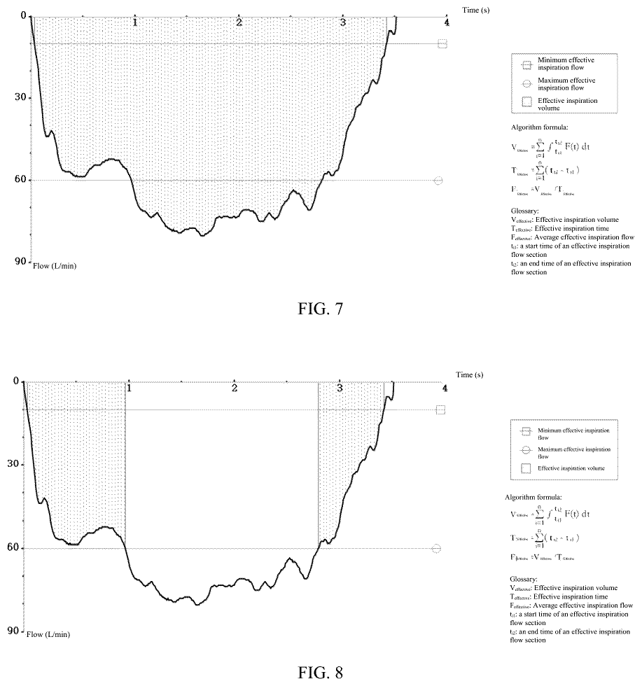

Test example 1 shows an inhalation evaluation test conducted by a user to simulate inhalation administration device Respimat® in a single-evaluation mode. Respimat® is an aerosol; a resistance gear thereof is 0, a lower limit of effective inspiration flow thereof is 10 L/min, an upper limit of effective inspiration flow thereof is 60 L/min, an ideal inspiration flow thereof is 30 L/min, and a recommended breath-holding time thereof is 10 seconds after inspiration. Mode 1 is selected for the analysis method, that is, the analysis method based on a lower limit of effective inspiration flow without considering an upper limit thereof. A flow-time curve and an analysis diagram in the test example are shown in FIG. 7, and the analysis results are shown in Table 3. As shown in a shaded area of the curve in FIG. 7, based on the analysis method, a section that the user adopts the effective inspiration flow of Respimat® is very large, occupying most of an inspiration process. As shown in the results in Table 3, the effective inspiration volume ratio of Respimat® inspiration simulated by the user is as high as 99.4%, the average effective inspiration flow is 60.6 L/min, and the breath-holding time is greater than or equal to 10 seconds. It can be seen that, regardless of the upper limit of effective inspiration flow, the effective inspiration volume ratio of Respimat® used by the user is very good, but the average effective inspiration flow is far from the ideal inspiration flow. According to parameters such as the effective inspiration volume ratio of the tester, a doctor can determine whether it is appropriate for a tester to use Respimat® as the aerosol and select an appropriate inhalation administration device for the tester.

TABLE 3
Analysis results of test example 1

| Indicator | Result |
|---|---|
| Peak inspiration flow (L/min) | 81.6 |
| Total inspiration volume (L) | 3.44 |
| Effective inspiration volume (L) | 3.42 |
| Effective inspiration volume ratio (%) | 99.4 |
| Total inspiration duration (sec.) | 3.51 |
| Effective inspiration duration (sec.) | 3.38 |
| Average effective inspiration flow (L/min) | 60.6 |
| Breath-holding duration after inspiration (sec.) | ≥10 |

### TEST EXAMPLE 2

Test example 2 analyzes the flow-time curve result of test example 1 in a mode 2, that is, the analysis method that both the lower limit of effective inspiration flow and the upper limit of effective flow are considered, and the flow-time curve and analysis diagram thereof are shown in FIG. 8, with analysis results shown in Table 4. It can be seen from FIG. 8 that an effective inspiration flow section (a shaded area) is divided into two sections by an intermediate part exceeding the upper limit of effective inspiration flow, and the intermediate part exceeding the limit occupies a large part of the sections. It can be seen from Table 4 that, based on the analysis mode, the effective inspiration volume ratio of Respimat® inspiration simulated by the user is only 34.6%, the average effective inspiration flow is 46.2 L/min, and the breath-holding time is greater than or equal to 10 seconds. It can be seen that, considering the upper limit of effective inspiration flow, the effective inspiration volume ratio of Respimat® inspiration simulated by the user is not high (34.6%), and a large part of inhalation is because the inhalation flow is too high and exceeds the upper limit of effective inhalation flow, which may lead to the failure of effective deposition of drugs at a target site, resulting in the waste of drugs and affecting the administration effect. According to parameters such as the effective inspiration volume ratio of the tester, a doctor can select an appropriate inhalation administration device for the tester.

TABLE 4
Analysis results of test example 2

| Indicator | Result |
|---|---|
| Peak inspiration flow (L/min) | 81.6 |
| Total inspiration volume (L) | 3.44 |
| Effective inspiration volume (L) | 1.19 |
| Effective inspiration volume ratio (%) | 34.6 |
| Total inspiration duration (sec.) | 3.51 |
| Effective inspiration duration (sec.) | 1.55 |
| Average effective inspiration flow (L/min) | 46.2 |
| Breath-holding duration after inspiration (sec.) | ≥10 |

### TEST EXAMPLE 3

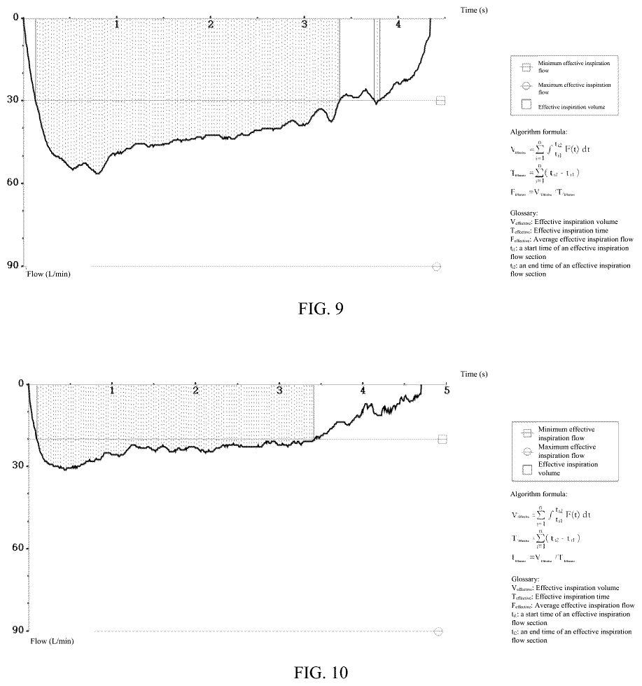

Test example 3 shows an inhalation evaluation test conducted by a user to simulate inhalation administration device Accuhaler® (Diskus®) in a single-evaluation mode. For Accuhaler® (Diskus®), a resistance gear thereof is 2, a lower limit of effective inspiration flow thereof is 30 L/min, an upper limit of effective inspiration flow thereof is 90 L/min, an ideal inspiration flow thereof is 60 L/min, and a recommended breath-holding time thereof is 10 seconds after inspiration. Mode 2 is selected for the analysis method, that is, the analysis method based on a lower limit of effective inspiration flow and an upper limit of effective inspiration flow. A flow-time curve and an analysis diagram in the test example are shown in FIG. 9, and the analysis results are shown in Table 5. As shown in FIG. 9, the inspiration flow of the resistance gear used by the user is significantly lower than the upper limit of effective inspiration flow of the simulated inhalation administration device Accuhaler® (Diskus®), so the effective inspiration flow section is really only whether lower limit of effective inspiration flow is reached. It can be seen from Table 5 that, the effective inspiration volume ratio of Accuhaler® (Diskus®) inspiration is 90.5%, the average effective inspiration flow is 43.2 L/min, and the breath-holding time is greater than or equal to 10 seconds. It can be seen that the effective inspiration volume ratio of Accuhaler® (Diskus®) inspiration simulated by the user is very good, and the average effective inspiration flow and ideal inspiration flow thereof are still slightly different.

TABLE 5
Analysis results of test example 3

| Indicator | Result |
|---|---|
| Peak inspiration flow (L/min) | 57.0 |
| Total inspiration volume (L) | 2.85 |
| Effective inspiration volume (L) | 2.58 |
| Effective inspiration volume ratio (%) | 90.5 |
| Total inspiration duration (sec.) | 4.34 |
| Effective inspiration duration (sec.) | 3.60 |
| Average effective inspiration flow (L/min) | 43.2 |
| Breath-holding duration after inspiration (sec.) | ≥10 |

### TEST EXAMPLE 4

Test example 4 shows an inhalation evaluation test conducted by a user to simulate inhalation administration device Handilaler® in a single-evaluation mode. Handilaler® has relatively great resistance, a resistance gear thereof is 5, a lower limit of effective inspiration flow thereof is 20 L/min, an upper limit of effective inspiration flow thereof is 90 L/min, an ideal inspiration flow thereof is 30 L/min, and a recommended breath-holding time thereof is 10 seconds after inspiration. Mode 2 is selected for the analysis method, that is, the analysis method based on a lower limit of effective inspiration flow and an upper limit thereof. A flow-time curve and an analysis diagram in the test example are shown in FIG. 10, and the analysis results are shown in Table 6. As shown in FIG. 10, the inspiration flow of the resistance gear used by the user is far lower than the upper limit of effective inspiration flow of the simulated inhalation administration device Handilaler , so the effective inspiration flow section is really only whether lower limit of effective inspiration flow is reached. It can be seen from Table 6 that, the effective inspiration volume ratio of Handilaler® inspiration is 84.0%, the average effective inspiration flow is 24.6 L/min, and the breath-holding time is greater than or equal to 10 seconds. It can be seen that the effective inspiration volume ratio of Handilaler® inspiration simulated by the user is very good, but the internal resistance of Handilaler® is relatively large, and the average effective inspiration flow and ideal inspiration flow thereof are still slightly different.

TABLE 6
Analysis results of test example 4

| Indicator | Result |
|---|---|
| Peak inspiration flow (L/min) | 32.4 |
| Total inspiration volume (L) | 1.63 |
| Effective inspiration volume (L) | 1.37 |
| Effective inspiration volume ratio (%) | 84.0 |
| Total inspiration duration (sec.) | 4.69 |
| Effective inspiration duration (sec.) | 3.33 |
| Average effective inspiration flow (L/min) | 24.6 |
| Breath-holding duration after inspiration (sec.) | ≥10 |

It can be seen that a system for evaluating inhalation administration proposed in the present invention provides an evaluation device and an evaluating and analysis method based on inspiration flow-time data of patients, and proposes and provides an effective inspiration duration (T_{effective}), an effective inspiration volume (V_{effective}), an average effective inspiration flow (F_{effective}), a total inspiration volume (Vₜₒₜₐₗ), an effective inspiration volume ratio ((E_{effective}), an ideal inspiration volume (V_{ideal}), an ideal inspiration volume ratio (V_{ideal}), a breath-holding duration (T_{breath-holding}), a peak inspiration flow (PIF) and the like, as well as parameters related to inhalation administration quality and calculation analysis methods thereof. An evaluation test of whether the inhalation administration device matches with patients is quantified, facilitating accurate evaluation by a user, and solving the problem the prior art that the inhalation administration device being suitable for the patients cannot be effectively and accurately evaluated, which can accurately select the inhalation administration device, improve the quality of medication, and reduce the waste of medication.

1. A method for evaluating inhalation administration, comprising the following steps: 1) using a flow detector and an inhalation administration device to be evaluated or a corresponding simulator to perform measurement according to inspiration requirements for the inhalation administration device, to obtain data of an inspiration flow-time curve (F-T); 2) according to an inspiration flow parameter of the inhalation administration device, obtaining a start time tᵢ₁ and an end time tᵢ₂ of each effective inspiration flow section on the inspiration flow-time curve; 3) calculating an effective inspiration volume V_{effective} based on the inspiration flow-time curve and a series of time parameters tᵢ₁ and tᵢ₂ obtained; and 4) evaluating an effective inhalation state of a user using the inhalation administration device with the effective inspiration volume V_{effective} or indicators associated with the effective inspiration volume V_{effective}, to determine whether the inhalation administration device is applicable to the user.

2. The method according to claim 1, wherein the inspiration flow parameter comprises a lower limit of effective inspiration flow.

3. The method according to claim 2, wherein a method for determining the effective inspiration flow section comprises: taking a time point t₁₁ when inspiration flow reaches a preset lower limit of the effective inspiration flow for the first time as a start time of a first effective inspiration flow section; taking a time point t₁₂ when the inspiration flow drops to the preset lower limit of the effective inspiration flow for the first time as an end time of the first effective inspiration flow section; taking t₁₁ and t₁₂ sections in the inspiration flow-time curve as the effective inspiration flow section; and obtaining all effective inspiration flow sections by analogy.

4. The method according to claim 1, wherein the inspiration flow parameter comprises a lower limit of effective inspiration flow and an upper limit thereof.

5. The method according to claim 4, wherein a method for determining the effective inspiration flow section comprises: taking a time point t₁₁ when an inspiration flow reaches a preset lower limit of the effective inspiration flow for the first time as a start time of a first effective inspiration flow section; taking a time point t₁₂ when the inspiration flow is beyond a range from the preset lower limit of the effective inspiration flow to a preset upper limit thereof for the first time as an end time of the first effective inspiration flow section; taking a time point t₂₁ when the inspiration flow starts to come into the range from the preset lower limit of the effective inspiration flow to the preset upper limit thereof for the second time as a start time of a second effective inspiration flow section; taking a time point t₂₂ when the inspiration flow is beyond the range from the preset lower limit of the effective inspiration flow to the preset upper limit thereof for the second time as an end time of the second effective inspiration flow section; and obtaining all effective inspiration flow sections by analogy.

6. The method according to claim 1, wherein the indicator associated with the effective inspiration volume E_{effective} is an effective inspiration volume ratio E_{effective}; the effective inspiration volume ratio E_{effective} is calculated by the following method:
obtaining an inspiration start time t₀ and an inspiration end time tₓ on the inspiration flow-time curve; calculating a total inspiration volume Vₜₒₜₐₗ based on the inspiration flow-time curve and the obtained time parameters t₀ and tₓ; and then calculating the effective inspiration volume ratio E_{effective}; wherein the total inspiration volume Vₜₒₜₐₗ and the effective inspiration volume ratio E_{effective} are calculated according to the following formula:
Vₜₒₜₐₗ=∫ₜ₀^{tx}F(t)dt
E_{effective}=(V_{effective}/Vₜₒₜₐₗ)*100%
wherein t denotes time, F(t) denotes a time function of inspiration flow, t₀ denotes an inspiration start time, and tₓ denotes an inspiration end time.

7. The method according to claim 1, further comprising: using an effective inspiration duration T_{effective} and/or an average effective inspiration flow F_{effective}, wherein the effective inspiration duration T_{effective} and the average effective inspiration flow F_{effective} are calculated according to the following formula:
T_{effective}=Σᵢ₌₁ⁿ(ti2−ti1)
F_{effective}=V_{effective}/T_{effective}
i denotes the serial number of the effective inspiration flow section, and i=1 . . . n, where 1 denotes a first effective inspiration flow section and n denotes a last effective inspiration flow section; tᵢ₁ denotes a start time of the i(th) effective inspiration flow section; and tᵢ₂ denotes an end time of the i(th) effective inspiration flow section.

8. The method according to claim 1, further comprising: determining a breath-holding duration, wherein a method for determining the breath-holding duration comprises: obtaining a breath-holding end time tₕ on the inspiration flow-time curve and calculating the breath-holding duration (T_{breath-holding}) according to the following formula:
T_{breath-holding}=tₕ−tₓ
wherein tₕ denotes the breath-holding end time, and tₓ denotes an inspiration end time and a breath-holding start time.

9. The method according to claim 1, wherein the effective inspiration volume V_{effective} is calculated according to the following formula:
V_{effective}=Σᵢ₌₁ⁿ(∫ₜᵢ₁^{ti2}F(t)dt)
t denotes time;
F(t) denotes a time function of inspiration flow;
i denotes the serial number of the effective inspiration flow section, and i=1 . . . n, wherein 1 denotes a first effective inspiration flow section and n denotes a last effective inspiration flow section;
tᵢ₁ denotes a start time of the i(th) effective inspiration flow section; and
tᵢ₂ denotes an end time of the i(th) effective inspiration flow section.

10. A system for evaluating inhalation administration, comprising a flow detector and an intelligent terminal, wherein the flow detector detects patient-related data of an inspiration flow-time curve (F-T) with a time change, and the intelligent terminal comprises a data processing system and a method for evaluating inhalation administration, the method is the method according to claim 1; a wire or wireless connection is adopted between the flow detector and the intelligent terminal; and inspiration flow-time data detected by the flow detector is uploaded to the intelligent terminal.

11. The system for evaluating inhalation administration according to claim 10, wherein the flow detector is equipped with an adjustable intake resistance unit and comprises different resistance gears.

12. The system for evaluating inhalation administration according to claim 10, wherein the inspiration flow parameter comprises a lower limit of effective inspiration flow.

13. The system for evaluating inhalation administration according to claim 12, wherein a method for determining the effective inspiration flow section comprises: taking a time point t₁₁ when inspiration flow reaches a preset lower limit of the effective inspiration flow for the first time as a start time of a first effective inspiration flow section; taking a time point t₁₂ when the inspiration flow drops to the preset lower limit of the effective inspiration flow for the first time as an end time of the first effective inspiration flow section; taking t₁₁ and t₁₂ sections in the inspiration flow-time curve as the effective inspiration flow section; and obtaining all effective inspiration flow sections by analogy.

14. The system for evaluating inhalation administration according to claim 10, wherein the inspiration flow parameter comprises a lower limit of effective inspiration flow and an upper limit of effective inspiration flow.

15. The system for evaluating inhalation administration according to claim 4, wherein a method for determining the effective inspiration flow section comprises: taking a time point t₁₁ when inspiration flow reaches a preset lower limit of the effective inspiration flow for the first time as a start time of a first effective inspiration flow section; taking a time point t₁₂ when the inspiration flow is beyond a range from the preset lower limit of the effective inspiration flow to a preset upper limit thereof for the first time as an end time of the first effective inspiration flow section; taking a time point t 21 when the inspiration flow starts to come into the range from the preset lower limit of the effective inspiration flow to the preset upper limit thereof for the second time as a start time of a second effective inspiration flow section; taking a time point t₂₂ when the inspiration flow is beyond the range from the preset lower limit of the effective inspiration flow to the preset upper limit thereof for the second time as an end time of the second effective inspiration flow section; and obtaining all effective inspiration flow sections by analogy.

16. The system for evaluating inhalation administration according to claim 10, wherein the indicator associated with the effective inspiration volume_{effective} is an effective inspiration volume ratio E_{effective}; the effective inspiration volume ratio E_{effective} is calculated by the following method: obtaining an inspiration start time t₀ and an inspiration end time tₓ on the inspiration flow-time curve; calculating a total inspiration volume Vₜₒₜₐₗ based on the inspiration flow-time curve and the obtained time parameters t₀ and tₓ; and then calculating the effective inspiration volume ratio E_{effective}; wherein the total inspiration volume Vₜₒₜₐₗ and the effective inspiration volume ratio E_{effective} are calculated according to the following formula:
Vₜₒₜₐₗ=Σₜ₀^{tx}F(t)dt
E_{effective}=(V_{effective}/Vₜₒₜₐₗ)*100%
wherein t denotes time, F(t) denotes a time function of inspiration flow, t₀ denotes an inspiration start time, and tₓ denotes an inspiration end time.

17. The system for evaluating inhalation administration according to claim 10, wherein the method further comprises: using an effective inspiration duration T_{effective} and/or an average effective inspiration flow F_{effective} to determine whether an inhalation administration device is applicable to a patient, and calculating the effective inspiration duration T_{effective} and the average effective inspiration flow F_{effective} according to the following formula:
T_{effective}=Σᵢ₌₁ⁿ(ti2−ti1)
F_{effective}=V_{effective}/T_{effective}
i denotes the serial number of the effective inspiration flow section, and i=1 . . . n, where 1 denotes a first effective inspiration flow section and n denotes a last effective inspiration flow section; tᵢ₁ denotes a start time of the i(th) effective inspiration flow section; and tᵢ₂ denotes an end time of the i(th) effective inspiration flow section.

18. The system for evaluating inhalation administration according to claim 10, wherein the method further comprises: determining a breath-holding duration, wherein a method for determining the breath-holding duration comprises: obtaining a breath-holding end time tₕ on the inspiration flow-time curve and calculating the breath-holding duration (T_{breath-holding}) according to the following formula:
T_{breath-holding}=tₕ−tₓ
wherein tₕ denotes the breath-holding end time, and tₓ denotes an inspiration end time and a breath-holding start time.

19. The system for evaluating inhalation administration according to claim 10, wherein an effective inspiration volume V_{effective} is calculated according to the following formula:
V_{effective}=Σᵢ₌₁ⁿ(∫ₜᵢ₁^{ti2}F(t)dt)
t denotes time;
F(t) denotes a time function of inspiration flow;
i denotes the serial number of the effective inspiration flow section, and i=1 . . . n, wherein 1 denotes a first effective inspiration flow section and n denotes a last effective inspiration flow section;
tᵢ₁ denotes a start time of the i(th) effective inspiration flow section; and
tᵢ₂ denotes an end time of the i(th) effective inspiration flow section.

20. The system for evaluating inhalation administration according to claim 10, wherein the intelligent terminal further comprises an evaluation guiding module for guiding an evaluation test of a patient, and the evaluation guiding module comprises one or more guidance forms of sounds, characters, graphics and animations.
